(19) United States
(12) Patent Application Publication
(10) Pub. No.: US 2016/0296451 A1
(43) Pub. Date: Oct. 13, 2016

(54) Edible Aqueous Nail Polish, and New Use for Edible Exudate Gum

(71) Applicant: Joshua D'Amico Kroot, San Francisco, CA (US)

(72) Inventor: Joshua D'Amico Kroot, San Francisco, CA (US)

(21) Appl. No.: 15/097,279

(22) Filed: Apr. 12, 2016

Related U.S. Application Data
(60) Provisional application No. 62/146,388, filed on Apr. 12, 2015.

(51) Int. Cl. A61K 8/73; A61Q 3/02

(52) U.S. Cl. CPC A61K 8/73; A61Q 3/02; A61K 2800/20

(57) ABSTRACT

In one set of embodiments, an edible binder containing exudate gum is combined with colorant to form a novel edible aqueous nail polish that can be applied to human fingernails and toenails. In various embodiments, the nail polish and/or exudate gum is combined with various solvents, thickeners, preservatives and other materials. Other embodiments are described and shown.

### CROSS-REFERENCE TO RELATED APPLICATIONS

This application claims the benefit of provisional patent application No. 62146388, filed 2015 Apr. 12 by the present inventor.

### FEDERALLY SPONSORED RESEARCH

Not applicable.

### SEQUENCE LISTING OR PROGRAM

Not applicable.

### BACKGROUND

This relates to nail polish, specifically edible, aqueous nail polish.

The following is a tabulation of some prior art that presently appears relevant:

U.S. Patents

| Patent Nr. | Kind Code | Issue Date | Patentee |
|---|---|---|---|
| 5,965,111 | A | 1999 Oct. 12 | Ellingson et al. |

U.S. Patent Application Publications

| Publication Nr. | Kind Code | Publication Date | Applicant |
|---|---|---|---|
| 2005/0255063 | A1 | 2005 Nov. 17 | Fenwick-Le Vine |

Foreign Patent Documents

| | Country | | | |
|---|---|---|---|---|
| Foreign Doc. Nr. | Code | Kind Code | Pub. Date | App. or Patentee |
| 19900106315 | EP | B1 | 1993 Sep. 8 | Koch et al. |
| 2976398 | EP | A1 | 2016 Jan. 27 | Anne |
| 100346770 | CN | C | 2007 Nov. 7 | [symbol] et al. |

Most commercial nail polishes contain chemicals known to cause harm to human health, in concentrations sufficient to cause such harm, for example dibutyl phthalate, formaldehyde, methylene glycol, formalin, toluene, tosylamide, camphor and acetone. Removal of conventional nail polish is usually accomplished by applying a nail polish remover, which generally also contains acetone. These chemicals can be absorbed through skin and nails. They can also enter the body through the respiratory system, for example when a person breathes gases or aerosols released from nail polish or nail polish remover. They can enter the body through the digestive tract as well, particularly when nail polish chips or fragments during food preparation or consumption, or when a person bites his or her nails.

Consequently, health professionals, nail polish consumers, manufacturers, retailers and others have expressed concern over the potential harmful effects of nail polish and nail polish remover on the human body and on the natural environment. Of particular concern are the effects on children, expectant mothers, salon workers, people with chemical sensitivities, and those undergoing certain medical treatments such as chemotherapy.

Consequently, several nail polishes have come to market that label themselves as being free of some of the most potentially harmful chemicals that are often contained in nail polish. However, they still contain significant quantities of other potentially harmful chemicals, and they are still generally acetone-based and/or removed with an acetone-based remover.

Certain nail polishes have therefore been designed to contain little or no acetone, including some nail polishes that label themselves as “aqueous-based” or “water-based”. Even these nail polishes, however, contain ingredients that are not approved for ingestion by humans. They are therefore still potentially problematic if they chip or otherwise enter the digestive system. This is of particular concern for children, who often place their hands in their mouths. These nail polishes are also not removable solely with water. They generally require a remover containing acetone, alcohol, borax, or other potentially toxic chemicals that are not suitable for use by children, expectant mothers and people with chemical sensitivities.

U.S. Publication Nr. 2005/0255063 by Fenwick-Le Vine purports to disclose a nail polish comprising a water solvent and an edible shellac. However, shellac is not soluble in water unless borax or another corrosive, non-edible base material is added to the water, resulting in an article that is neither edible nor non-toxic to humans. Furthermore, such a shellac requires either an alcohol-based remover or a corrosive non-edible base-based remover, neither of which is suitable for use by children, expectant mothers and people with chemical sensitivities.

European Publication Nr. 2976398 discloses a non-edible nail polish comprising a food material. However, the food material is added to a filmogen agent that is non-edible, and that in the embodiments described in the publication, contains chemicals known to cause harm to human health in sufficient concentrations to cause such harm.

Several aqueous nail polishes and/or methods of preparation have also been proposed, including U.S. Pat. No. 5,965,111, European Patent Nr. 19,900,106,315 and Chinese Patent Nr. 100346770. However, none of these polishes is edible. Furthermore, the constituents of U.S. Pat. No. 5,965,111 include volatile organic solvents that are not only inedible, but that in significant concentrations are acutely toxic.

The present inventor has surprisingly discovered that an edible binder containing exudate gum can be used to create a variety of applications that behave in a manner similar to conventional nail polish, but that are edible and can be removed solely with water.

### DRAWINGS

Not applicable.

### DETAILED DESCRIPTION

### First Embodiment of Edible Aqueous Nail Polish

One embodiment is composed of an edible binder containing exudate-gum and one or more edible colorants. The binder and the colorant or colorants can be fully or partially intermixed with each other. “Edible” means any material that can be ingested by any human, and/or any material that can be sold, donated, gifted and/or otherwise given to any human for ingestion, either alone or together with other materials, under the laws and/or regulations of any country and/or territory of the world. “Exudate gum” means any material excreted from a plant that is partially or fully soluble in water and solid or semi-solid when dry. Examples include, but are not limited to, acacia fiber, acacia gum, gum arabic, acacia senegal gum, gum talha, gum ghatti, anogeissus latifolia gum, karroo gum, acacia karroo gum, feronia limonia gum, cochlospermum religiosum gum, gum karaya, sterculia gum, gum kondagogu, cochlospermum gum, gum tragacanth, and astragalus gum. “Binder” means any material that, when in liquid form, or when fully or partially dissolved in solution, adheres or partially adheres to a surface to which it is applied. “Colorant” means any material that changes the color of any other material and/or combination of materials to which it is added and/or applied.

In one embodiment, the binder is an edible acacia fiber (gum arabic) powder, such as Acacia Fiber Organic Powder—available from NOW Health Group, Inc. of Bloomingdale, Ill. However, the binder can consist of any other edible material containing and/or derived from one or more exudate gums which, when fully or partially dissolved in solution, adheres or partially adheres to a surface to which it is applied. “Solution” means either or both of: (1) any combination of any material with any liquid and/or gas in which some or all of the material is fully and/or partially dissolved (the combination can also include material that is not dissolved, and/or material that is suspended in the liquid and/or gas); and/or (2) any material or combination of materials some or all of which was at one time dissolved or suspended in liquid and/or gas, even if the matter that comprised the liquid and/or gas in no longer present or is present in a diminished amount in the material or combination of materials (for example because some or all of the liquid has evaporated and/or some or all of the gas has condensed).

In one embodiment, the colorant is an edible juice powder, such as Beet Juice Powder, available from Pines International, Inc. of Lawrence, Kans. However, the colorant or colorants can consist of any material that is edible, water soluble, and gives color to, and/or changes the color of, the embodiment. Examples include, but are not limited to, various solid or partially-solid materials derived from biological organisms and/or parts thereof, such as spinach, chlorella, raspberry, blueberry, eggplant, orange, saffron, turmeric, annatto, mushroom, milk, egg and/or cochineal. Further examples of colorants include, but are not limited to, various liquid or partially-liquid materials derived from biological organisms and/or parts thereof, such as beet juice, orange juice, milk, egg, honey, and/or kombucha. Other examples include, but are not limited to, edible color additives such as the color additives listed in the United States Food and Drug Administration Regulations (United Code of Federal Regulations, Title 21). “Water soluble” means any material capable of partially or fully dissolving in distilled water.

### First Embodiment of New Use of Edible Exudate Gum

One embodiment uses one or more edible exudate gums as a component of a binder in combination with one or more edible colorants to form a mixture that, in the presence of one or more solvents, creates an application that adheres to human fingernails and toenails. “Solvent” means any material that is capable of fully or partially dissolving another material and/or combination of materials. “Application” means any material and/or combination of materials that is capable of adhering or partially adhering to a surface. For the aforementioned embodiment, the solvent or solvents can be water, and/or a material containing water, in sufficient quantity to fully or partially dissolve the binder, and/or any portion thereof. The viscosity of the resulting application can be regulated by controlling the amount of solvent in the embodiment.

The application can be applied to one or more human fingernails and/or human toenails. Once the application has been so applied, it can be left undisturbed for the time required to allow the solvent to evaporate and/or partially evaporate until the application is no longer sticky to the touch. “Evaporate” means for a material and/or any portion of a material to change from being in a liquid phase to being in a gaseous phase.

### Alternative Embodiments of Edible Aqueous Nail Polish

In one alternative embodiment, an anti-caking agent is included in the embodiment, in addition to the binder and/or colorants and/or other material(s) comprising some or all of any embodiment or any part of any embodiment. The anti-caking agent can be fully or partially intermixed with some or all of the other material(s) comprising some or all of any embodiment or any part of any embodiment, and/or it can be fully or partially separated from some or all of the other material(s) comprising some or all of any embodiment or any part of any embodiment. The anti-caking agent can be edible corn starch, such as Argo Corn Starch, available from ACH Food Companies, Inc. of Cordova, Tenn. However, the anti-caking agent can be any material that is edible and prevents certain particles in the embodiment from adhering to certain other particles in the embodiment. Examples of ways in which the anti-caking agent may accomplish this include, but are not limited to, absorbing liquid, coating particles thereby making them resistant to liquid, and/or providing lubrication between particles.

In another alternative embodiment, thickener is included in the embodiment, in addition to the binder and/or colorants and/or other material(s) comprising some or all of any embodiment or any part of any embodiment. The thickener can be fully or partially intermixed with some or all of the other material(s) comprising some or all of any embodiment or any part of any embodiment, and/or it can be fully or partially separated from some or all of the other material(s) comprising some or all of any embodiment or any part of any embodiment. The thickener can be edible corn starch, such as Argo Corn Starch, available from ACH Food Companies, Inc. of Cordova, Tenn. However, the thickener can be any material that is edible and increases the viscosity of the embodiment in the presence of solvent.

In another alternative embodiment, solvent is included in the embodiment, in addition to the binder and/or colorants and/or other material(s) comprising some or all of any embodiment or any part of any embodiment. The solvent can be fully or partially intermixed with some or all of the other material(s) comprising some or all of any embodiment or any part of any embodiment, and/or it can be fully or partially separated from some or all of the other material(s) comprising some or all of any embodiment or any part of any embodiment.

In another alternative embodiment, the binder is liquid or partially liquid. “Liquid” means the state and/or phase of any material or combination of materials in which the material or combination of materials is capable of flowing; solid material and/or a combination of solid materials may form all or part of a liquid. Examples of materials in liquid form include, but are not limited to, distilled water whose temperature is above 0° C. and below 100° C. at sea level and/or ethanol whose temperature is above −114° C. and below 78.5° C. at sea level. Examples of liquids in which solid material and/or a combination of solid materials forms all or part of a liquid include, but are not limited to, suspensions such as orange juice and or groups of pieces of solid material such as sand and/or gravel.

In another alternative embodiment, the colorant or colorants are liquid or partially liquid.

In another alternative embodiment, a preservative is included in the embodiment, in addition to the binder and/or colorants and/or other material(s) comprising some or all of any embodiment or any part of any embodiment. In one embodiment, the preservative is edible citric acid, such as Citric Acid, available from NOW Health Group, Inc. of Bloomingdale, Ill. However, the preservative can be any edible material that decreases the rate of decay of the some or all of the binder, and/or of the colorants, and/or of the solvent, and/or of any other material comprising some or all of any embodiment or any part of any embodiment. “Decay” means any change to any embodiment, and/or any portion of any embodiment, and/or any material contained in any embodiment, that makes it less suitable for the operation of the embodiment, as described herein, than it was before the change. Examples of decay include, but are not limited to chemical changes brought about by exposure to heat, cold, vibration, light, and/or other radiation. Other examples of decay include, but are not limited to, physical changes such as those brought about by heat, cold, vibration, light, and/or other radiation, such as the evaporation and/or release of liquid from any embodiment, and/or any portion of any embodiment, and/or any material contained in any embodiment. Other examples of decay include, but are not limited to, physical and/or chemical changes brought about by consumption and/or colonization of any embodiment, and/or any portion of any embodiment, and/or any material contained in any embodiment by any living organism, such as bacteria, protists, fungi, plants, insects, worms and/or other animals.

In another alternative embodiment, the embodiment is heated in the presence of solvent to a temperature sufficient to incapacitate micro-organisms commonly responsible for food borne illness and/or food spoilage. Such micro-organisms may include, but are not limited to, bacteria, viruses, yeasts, molds, protozoa and/or prions.

In another alternative embodiment, the embodiment is heated in the presence of solvent to a temperature sufficient to cause the thickening of the embodiment.

### Alternative Embodiments of New Use of Edible Exudate Gum

In one alternative embodiment, an anti-caking agent is included in the embodiment, in addition to the binder and/or colorants and/or other material(s) comprising some or all of any embodiment or any part of any embodiment. The anti-caking agent can be fully or partially intermixed with some or all of the other material(s) comprising some or all of any embodiment or any part of any embodiment, and/or it can be fully or partially separated from some or all of the other material(s) comprising some or all of any embodiment or any part of any embodiment. The anti-caking agent can be edible corn starch, such as Argo Corn Starch, available from ACH Food Companies, Inc. of Cordova, Tenn. However, the anti-caking agent can be any material that is edible and prevents certain particles in the embodiment from adhering to certain other particles in the embodiment. Examples of ways in which the anti-caking agent may accomplish this include, but are not limited to, absorbing liquid, coating particles thereby making them resistant to liquid, and/or providing lubrication between particles.

In another alternative embodiment, thickener is included in the embodiment, in addition to the binder and/or colorants and/or other material(s) comprising some or all of any embodiment or any part of any embodiment. The thickener can be fully or partially intermixed with some or all of the other material(s) comprising some or all of any embodiment or any part of any embodiment, and/or it can be fully or partially separated from some or all of the other material(s) comprising some or all of any embodiment or any part of any embodiment. The thickener can be edible corn starch, such as Argo Corn Starch, available from ACH Food Companies, Inc. of Cordova, Tenn. However, the thickener can be any material that is edible and increases the viscosity of the embodiment in the presence of solvent.

In another alternative embodiment, solvent is included in the embodiment, in addition to the binder and/or colorants and/or other material(s) comprising some or all of any embodiment or any part of any embodiment. The solvent can be fully or partially intermixed with some or all of the other material(s) comprising some or all of any embodiment or any part of any embodiment, and/or it can be fully or partially separated from some or all of the other material(s) comprising some or all of any embodiment or any part of any embodiment.

In another alternative embodiment, the binder is liquid or partially liquid. “Liquid” means the state and/or phase of any material or combination of materials in which the material or combination of materials is capable of flowing; solid material and/or a combination of solid materials may form all or part of a liquid. Examples of materials in liquid form include, but are not limited to, distilled water whose temperature is above 0° C. and below 100° C. at sea level and/or ethanol whose temperature is above −114° C. and below 78.5° C. at sea level. Examples of liquids in which solid material and/or a combination of solid materials forms all or part of a liquid include, but are not limited to, suspensions such as orange juice and or groups of pieces of solid material such as sand and/or gravel.

In another alternative embodiment, the colorants are liquid or partially liquid.

In another alternative embodiment, a preservative is included in the embodiment, in addition to the binder and/or colorants and/or other material(s) comprising some or all of any embodiment or any part of any embodiment. In one embodiment, the preservative is edible citric acid, such as Citric Acid, available from NOW Health Group, Inc. of Bloomingdale, Ill. However, the preservative can be any edible material that inhibits the growth of micro-organisms commonly responsible for food borne illness, and/or decreases the rate of decay of the some or all of the binder, and/or of the colorants, and/or of the solvents, and/or of any other material comprising some or all of any embodiment or any part of any embodiment. “Decay” means any change to any embodiment, and/or any portion of any embodiment, and/or any material contained in any embodiment, that makes it less suitable for the operation of the embodiment, as described herein, than it was before the change. Examples of decay include, but are not limited to chemical changes brought about by exposure to heat, cold, vibration, light, and/or other radiation. Other examples of decay include, but are not limited to, physical changes such as those brought about by heat, cold, vibration, light, and/or other radiation, such as the evaporation and/or release of liquid from any embodiment, and/or any portion of any embodiment, and/or any material contained in any embodiment. Other examples of decay include, but are not limited to, physical and/or chemical changes brought about by consumption and/or colonization of any embodiment, and/or any portion of any embodiment, and/or any material contained in any embodiment by any living organism, such as bacteria, protists, fungi, plants, insects, worms and/or other animals.

In another alternative embodiment, the embodiment is heated in the presence of solvent to a temperature sufficient to incapacitate micro-organisms commonly responsible for food borne illness and/or decay. Such micro-organisms may include, but are not limited to, bacteria, viruses, yeasts, molds, protozoa and/or prions.

In another alternative embodiment, the embodiment is heated in the presence of solvent to a temperature sufficient to cause the thickening of the embodiment.

### Conclusions, Ramifications, and Scope

Accordingly, the reader will see that at least one embodiment provides a nail polish that significantly reduces the risk of toxicity associated with the application, use and removal of currently-existing nail polishes. Furthermore, the embodiments have the advantages that:
they can be applied, worn and removed by children with minimal supervision;
they can be used by pregnant women and nursing mothers without endangering their own health or that of their children;
they are suitable for use by occupational therapists and other medical professionals who work with patients who may be inclined to ingest nail polish;
they can be used by people undergoing chemotherapy and those with chemical sensitivities;
they can be used by salon workers without endangering their health or that of their clients;
they can be removed with just water, negating the need for acetate-based or other potentially toxic remover;
they are safer for the environment and does not present a disposal hazard when disposed of in a conventional waste bin; and
they can be made entirely of food ingredients, dramatically improving its smell over that of other nail polishes.

Although the description above contains many specificities, these should not be construed as limiting the scope of the embodiments but as merely providing illustrations of some of several embodiments. For example, one or more embodiments described above could be combined and/or varied. The scope of the embodiments should therefore not be limited by the above described embodiments and/or examples, but by all embodiments within the scope and spirit of the foregoing written description.

I claim:

1. An article for application to fingernails and toenails for the purpose of decorating said fingernails and said toenails, comprising a mixture of:
a. an edible binder containing exudate gum, and
b. edible colorant mixed with said binder,
whereby said mixture may be applied to said fingernails and said toenails for the purpose of decoration of said fingernails and said toenails.

2. The mixture of claim 1 wherein said mixture contains solvent selected from the group consisting of water and solvents containing water.

3. The mixture of claim 1 wherein said mixture contains a preservative in sufficient quantity to retard the growth of micro-organisms.

4. The mixture of claim 1 wherein said mixture is heated to a temperature sufficient to retard the growth of micro-organisms.

5. The mixture of claim 1 wherein said mixture includes a thickener.

6. A new use for edible exudate gum comprising mixing said edible exudate gum with edible colorant and solvent containing water in a ratio sufficient to allow the resulting mixture to adhere to fingernails and toenails, whereby said mixture may be applied to said fingernails and said toenails for the purpose of forming an edible decorative coating on said fingernails and said toenails.

7. The new use of claim 6 wherein said mixture contains a preservative in sufficient quantity to retard the growth of micro-organisms.

8. The new use of claim 6 wherein said mixture is heated to a temperature sufficient to retard the growth of micro-organisms.

9. The new use of claim 6 wherein said mixture includes a thickener.
